(19) United States
(12) Patent Application Publication
(10) Pub. No.: US 2011/0190729 A1
(43) Pub. Date: Aug. 4, 2011

(54) METHODS OF INDUCING PLURIPOTENCY INVOLVING SOX2 PROTEIN

(75) Inventors: Mark Alexander Kirkland, Victoria (AU); Tamara Jane Gough, Victoria (AU)

(73) Assignee: CYTOMATRIX PTY LTD, Geelong, Victoria (AU)

(21) Appl. No.: 12/745,425

(22) PCT Filed: Nov. 28, 2008
(86) PCT No.: PCT/AU08/01764

§ 371 Date: Mar. 4, 2011

(30) Foreign Application Priority Data
Nov. 30, 2007 (AU) 2007906555

(51) Int. Cl. A61M 37/00; C12N 5/071; A61K 38/02; C12N 15/12; A61P 25/28; A61P 25/16; A61P 25/00; A61P 3/10; A61P 9/00; A61P 21/00; A61P 19/10; A61P 19/08

(52) U.S. Cl. 604/522; 435/377; 514/1.1; 435/455; 514/17.8; 514/17.9; 514/17.7; 514/7.3; 514/16.4; 514/16.9

(57) ABSTRACT

The invention relates to a method of inducing pluripotency in a responsive mammalian cell, which comprises introducing into the cell an effective amount for initiating pluripotency within the cell of Sox2 protein or a functionally equivalent analogue, variant or fragment thereof. The invention also relates to a method of treatment and/or prophylaxis of a degenerative disease or injury in a mammal, which comprises removing from the mammal one or more responsive cells and culturing the cells in a suitable medium, introducing into the cells an effective amount of Sox2 protein or a functionally equivalent analogue, variant or fragment thereof and subsequently returning the cells to the patient. In a further embodiment the invention relates to a method of treatment and/or prophylaxis of a degenerative disease or injury in a mammal, which comprises introducing into responsive cells of the mammal an effective amount of Sox2 protein or a functionally equivalent analogue, variant or fragment thereof.

### FIELD OF THE INVENTION

The present invention relates to methods of inducing pluripotency in mammalian cells which involve introducing into the cells Sox2 protein or a functionally equivalent analogue, variant or fragment thereof. The invention also relates to methods of generating pluripotent cell lines for subsequent use, for example, in investigation of the causes and treatments of diseases, in developing cells and tissues of various lineages for the testing of drugs and other therapies, and in developing differentiated cells for therapy. The methods of treatment involve the induction of pluripotency in cells that would otherwise be terminally differentiated or in stem cells of more limited potential (unipotent or multipotent). The methods can be conducted using the patient's own cells in vivo or in vitro, or using cells from an immunologically compatible donor, with the cells then being differentiated into desired cell types before being returned (or introduced, in the case of donor cells) to the patient.

### BACKGROUND TO THE INVENTION

Embryonic stem (ES) cells are pluripotent cells derived from the inner cell mass of the early blastocyst¹. Embryonic stem cells can be expanded in culture indefinitely, and can be induced to undergo differentiation along multiple lineages in vitro. These multiple lineages include tissues of all three germ layers (endoderm, mesoderm and ectoderm). When injected into a suitable host animal, ES cells can give rise to teratomas, tumours that include multiple mature tissue types representing all three germ layers. Furthermore, when mouse ES cells are introduced into a developing blastocyst, the introduced cells contribute to all tissues in the developing embryo, and if these embryos (known as chimeras) are allowed to develop into adult mice and are cross bred, pups are generated which are genetically identical to the introduced ES cells, demonstrating that these cells are truly pluripotent.

ES cells are therefore seen has having tremendous therapeutic potential for the production of mature tissues for the therapy of a wide range of diseases, including stroke, spinal cord injury, liver damage and heart diseases amongst many others². Unfortunately, the application of ES cells to clinical therapeutics is limited for several reasons. One reason is the fact that an embryo is destroyed in the process of isolating the cells, which many regard as ethically unsound. A second reason is that the ES cell lines are immunologically identical only to the embryo from which they are derived. Mature cells introduced into another individual for therapeutic purposes would almost certainly be destroyed by the recipients' immune system.

A recent breakthrough has been made in this field by the identification of a group of four critical transcription factors that, when transfected together into mature cells, can induce those cells to de-differentiate into ES-like cells. Such cells are termed induced pluripotent stem cells, or iPS cells. In the case of the mouse, these four factors are Oct4, Sox2, Klf4 and c-myc³. Fibroblasts from a mature animal or, more efficiently, from an embryo, when transfected with these four genes can convert into cell lines that have been shown to have all the pluriopotency of ES cells derived from a blastocyst. They can give rise to tissues from all germ layers in vitro, can give rise to teratomas when injected into mice, can contribute to all tissues when injected into a blastocyst, and these chimeras can give rise to animals genetically identical to the ES-like cells when cross bred^{4,5}. Unfortunately, these second generation animals display a very high incidence of tumours, arising because of reactivation of the retrovirally inserted genes that gave rise to the iPS cells in the first instance. Alternative approaches to the induction of pluripotency are therefore required. The ability of these four factors to induce pluripotency in human cells has recently been confirmed⁶, while a second research group has also demonstrated the induction of pluripotency using a different combination of factors—Oct4, Sox2, Nanog and Lin28⁷.

Oct4 is a POU-homeodomain-containing transcription factor that has been shown to be critical in the induction and maintenance of the pluripotent stem cell state⁸. Down regulation of Oct4 expression in ES cells causes them to differentiate and lose their pluripotency. Oct4 is expressed at low levels in some adult stem cell populations.

Sox2 is a member of the Sox (SRY-related HMG box) gene family that encode transcription factors with a single HMG DNA-binding domain. Sox2 belongs to the SOX B1 subgroup, which also includes Sox1 and Sox3, based on homology within and outside the HMG box⁹. Sox2 marks the pluripotent lineage of the early mouse embryo, so that like Oct4 it is expressed in the ICM, epiblast, and germ cells.

Homeobox genes are key regulators of tissue identity and stem cell behaviour. As discussed above, Oct4 is a homeodomain protein that is critical in the maintenance of pluripotency, as is another homeoprotein, NANOG. Other homeoproteins can direct cell differentiation—for example, the pancreatic homeoprotein PDX-1 when transfected into hepatocytes using an adenovirus or similar means, can induce those cells to differentiate into pancreatic cells¹⁰. Similarly, the cardiac homeoprotein CSX1/Nkx2.5 can induce mesenchymal stem cells to adopt a cardiac fate following transfection¹¹.

The concensus homeodomain sequence also includes a transduction domain, Penetratin, allowing the protein to cross the cell membrane, and it has been suggested that some homeoproteins may act as cell-cell signalling molecules in the embryo¹². Other transduction domains, such as the HIV-TAT sequence, have also been used experimentally to aid transmembrane delivery of proteins.

The present inventors have now determined that it is possible to initiate the expression of key downstream target genes of Sox2, such as NANOG, by introducing into target cells an effective amount of Sox2 protein (preferably, but not necessarily, in conjunction with HIV-TAT protein to form Sox2-TAT protein) or a functionally equivalent analogue, variant or fragment thereof. While the role of Sox2 in the establishment and maintenance of pluripotency is well known, it has not been previously suggested that the introduction of Sox2 protein (as opposed to the transfection of the Sox2 gene) into cells, optionally in conjunction with other relevant transcription factors, could be efficient in inducing or maintaining pluripotency. It has also not been suggested that pluripotency could be sustained following exposure to Sox2 protein, and nor that the cells so exposed could be used to derive iPS cell lines.

It is with the above background that the present invention has been conceived.

### SUMMARY OF THE INVENTION

According to one embodiment of the present invention there is provided a method of initiating pluripotency in a responsive mammalian cell, which comprises introducing into the cell an effective amount for inducing pluripotency within the cell of Sox2 protein or a functionally equivalent analogue, variant or fragment thereof.

According to another embodiment of the present invention there is provided a method of inducing pluripotency in a responsive human cell, which comprises introducing into the cell an effective amount for initiating pluripotency within the cell of Sox2-TAT protein in conjunction with one or more other transcription factors selected from Oct4, Nanog, Klf4, Lin28 and c-myc.

The methods above may be conducted in vivo within a mammalian organism or may be conducted in vitro.

According to another embodiment of the present invention there is provided a method of treatment and/or prophylaxis of a degenerative disease or injury in a mammal, which comprises removing from the mammal one or more responsive cells and culturing the cells in a suitable medium, introducing into the cells an effective amount of Sox2 protein or a functionally equivalent analogue, variant or fragment thereof and subsequently returning the cells to the patient.

According to a further embodiment of the present invention there is provided a method of treatment and/or prophylaxis of a degenerative disease or injury in a mammal, which comprises introducing into responsive cells of the mammal an effective amount of Sox2 protein or a functionally equivalent analogue, variant or fragment thereof.

Preferably the Sox2 protein or a functionally equivalent analogue, variant or fragment thereof is introduced into the cells in conjunction with HIV-TAT protein, most preferably in the form of Sox2-TAT protein.

In a preferred aspect of the invention the Sox2 or Sox2-TAT protein or functionally equivalent analogue, variant or fragment thereof is introduced into the cells in conjunction with one or more other transcription factors. Preferred transcription factors include Oct4, Nanog, Lin28, Klf4 and/or c-myc.

In another preferred aspect of the present invention the Sox2 or Sox2-TAT protein or functionally equivalent analogue, variant or fragment thereof is introduced into the cells in conjunction with one or more other transcription factors such as Oct4, Nanog, Lin28, Klf4 and/or c-myc, (as recombinant proteins or by transfection) together with growth factors or growth promoting agents suitable for the maintenance of pluripotency. Such growth factors may include members of the fibroblast growth factor family, and in particular FGF4, as well as insulin-like growth factors and epidermal growth factors. The combination of Sox2-TAT and optionally other transcription factors or their functionally equivalent analogues, variants or fragments along with other optional components such as growth factors will for convenience be referred to herein as the “treatment agent”.

According to a still further embodiment of the present invention the responsive mammalian cells are mammalian cells other than pluripotent stem cells. Preferably the responsive mammalian cells are selected from one or more of hepatocytes, fibroblasts, endothelial cells, B cells, T cells, dendritic cells, keratinocytes, adipose cells, epithelial cells, epidermal cells, chondrocytes, cumulus cells, neural cells, glial cells, astrocytes, cardiac cells, oesophageal cells, skeletal muscle cells, skeletal muscle satellite melanocytes, hematopoietic cells, osteocytes, macrophages, monocytes, mononuclear cells or stem cells including embryonic stem cells, embryonic germ cells, adult brain stem cells, epidermal stem cells, skin stem cells, pancreatic stem cells, kidney stem cells, liver stem cells, breast stem cells, lung stem cells, muscle stem cells, heart stem cells, eye stem cells, bone stem cells, spleen stem cells, immune system stem cells, cord blood stem cells, bone marrow stem cells and peripheral blood stem cells.

In another preferred embodiment of the invention the treatment agent is introduced utilising detergent, bacterial toxin or electroporation, permeabilisation, lisosomal delivery or with the use of cell-permeant peptide vectors or polyethylene glycol (PEG), each of which are techniques well known in the art as described in Sambruck & Russell¹³, the disclosure of which is included herein in its entirety by way of reference. For example, bacterial toxin permeabilisation may utilise streptolysin O and cell-permeable peptide vectors may include antennapedia/penetratin TAT and signal-peptide based sequences.

The Sox2-TAT or optionally other transcription factors or their functionally equivalent analogues or variants may be produced recombinantly or may be isolated from mammalian cells.

According to another embodiment of the present invention there is provided an agent for initiating pluripotency in a responsive mammalian cell, which comprises Sox2 protein or a functionally equivalent analogue, variant or fragment thereof and one or more physiologically acceptable carriers and/or diluents. Such an agent may further comprise one or more other transcription factors and/or one or more permeabilisation agents and/or one or more growth factors or growth promoting agents suitable for maintaining pluripotency. Preferably the other transcription factors are selected from Oct4, Nanog, Lin28, Klf4 and/or c-myc, or their functionally equivalent analogues, variants or fragments.

### DESCRIPTION OF THE FIGURES

The present invention will be further described, by way of example only, with reference to the figures wherein:

FIG. 1 shows a Western Blot of a nuclear extract of CHO cells expressing the Sox2-TAT construct, and the washes and eluate from the Nickel column purification process

FIG. 2 shows a bar graph of luciferase measurements (relative luciferase units) in two cell lines stably transfected with the Sox2-TAT construct, then transfected with pGL4 vector containing the Nanog promoter sequence. Sox2#1 represents the Sox2 clone #1, Sox2#2 represents the Sox2 clone #2, pGL4.13 is the positive control vector, pGL4.20 is the vector without the Nanog promoter insert, vector is the vector only control and pGL4.20 nanog is the pGL4.20 vector containing Nanog promoter transfected into CHO Flp-In cells (with no Sox2 sequence).

FIG. 3 shows the amino acid sequence of the Sox2-TAT construct, wherein the Igκ secretory signal is shown underlined, the TAT sequence is shown with dashed underlining, the Sox2 sequence is highlighted in grey, the V5 epitope is double underlined and the Poly His tag is shown in normal text.

### DESCRIPTION OF SEQUENCE LISTINGS

The invention will be further described with reference to the sequence listings, where:

SEQ ID NO. 1 shows the amino acid sequence of human Oct4.

SEQ ID NO. 2 shows the amino acid sequence of human Sox2.

SEQ ID NO. 3 shows the amino acid sequence of human Nanog.

SEQ ID NO. 4 shows the amino acid sequence of human Klf4.

SEQ ID NO. 5 shows the amino acid sequence of human c-myc.

SEQ ID NO. 6 shows the amino acid sequence of human Lin28

SEQ ID NO. 7 shows the amino acid sequence of the Sox2-TAT construct.

SEQ ID NO. 8 shows the nucleic acid sequence of the Nanog promoter.

SEQ ID NO. 9 shows the nucleic acid sequence of the Sox2 forward primer.

SEQ ID NO. 10 shows the nucleic acid sequence of the Sox2-TAT forward primer.

SEQ ID NO. 11 shows the nucleic acid sequence of the Sox2 and Sox2-TAT Reverse primer.

SEQ ID NO. 12 shows the nucleic acid sequence of the Nanog forward primer.

SEQ ID NO. 13 shows the nucleic acid sequence of the Nanog reverse primer.

### DETAILED DESCRIPTION OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

In a broad aspect of the invention, and as mentioned above, there is provided a method of initiating pluripotency in a responsive mammalian cell. By the phrase “responsive mammalian cell”, it is intended to encompass mammalian cells other than pluripotent stem cells, which when subject to treatments according to the invention are seen to exhibit properties of pluripotency. For example, types of mammalian cells that may be treated according to the invention to initiate pluripotency include hepatocytes, fibroblasts, endothelial cells, B cells, T cells, dendritic cells, keratinocytes, adipose cells, epithelial cells, epidermal cells, chondrocytes, cumulus cells, neural cells, glial cells, astrocytes, cardiac cells, oesophageal cells, muscle cells, melanocytes, hematopoietic cells, osteocytes, macrophages, monocytes, mononuclear cells or stem cells including embryonic stem cells, embryonic germ cells, adult brain stem cells, epidermal stem cells, skin stem cells, pancreatic stem cells, kidney stem cells, liver stem cells, breast stem cells, lung stem cells, muscle stem cells, heart stem cells, eye stem cells, bone stem cells, mesenchymal stem cells, spleen stem cells, immune system stem cells, cord blood stem cells, bone marrow stem cells and peripheral blood stem cells. Of these cell types, there may be some that due to their cellular machinery and mechanisms may be preferred over others. For example, cell types adapted to produce other protein products or hormones may demonstrate particular suitability for the induction of pluripotency, when treated according to the invention. In particularly preferred embodiments of the invention the cells utilised are stem cells, as referred to above, mesenchymal stem cells, bone marrow stem cells or fibroblasts.

The cells utilised according to the invention may be derived from any of a variety of mammalian organisms, including, but not limited to humans, primates such as chimpanzees, gorillas, baboons, orangutans, laboratory animals such as mice, rats, guinea pigs, rabbits, domestic animals such as cats and dogs, farm animals such as horses, cattle, sheep, goats or pigs or captive wild animals such as lions, tigers, elephants, buffalo, deer or the like. In the treatment methods of the invention it is preferable, however, for cells used in treating a particular mammalian patient to be derived from an individual of the same species. Most preferably, and to minimise problems associated with immune rejection, cells used to treat a particular patient will be derived from the same patient.

By the phrase “inducing pluripotency” it is intended to convey that as a result of the treatment conducted at least some, preferably at least 0.01%, more preferably at least 0.1%, still more preferably at least 1%, particularly preferably at least 10% and more preferably at least 20, 30, 40, 60, 80 or 90% of the mammalian cells treated according to the invention will demonstrate features of pluripotency as a result of the treatment according to the invention. Cellular pluripotency may for example be detected by immunohistochemistry, by the use of specific stains for proteins usually expressed in ES cells or other detectable compounds, by radio-immunoassay or real time PCR which more particularly monitors stem cell gene expression. At least in the case of radio-immunoassay and real time PCR it is possible to quantify the levels of stem cell gene expression in a particular population of cells.

A key aspect of the present invention is the introduction into the cell or cells in which pluripotency is to be initiated of Sox2 protein or a functionally equivalent analogue, variant or fragment thereof. Sox2 is an HMG-box containing transcription factor known to be important in pluripotency. The Sox2 gene is localised on human chromosome 3 and the nucleotide sequence of the gene has been reported by Stevanovic et al¹⁴. Regulation of Sox2 gene expression is further described by Kamachi et al¹⁰, and its role in pluripotency has been described by Boiani and Scholer¹⁵ The disclosures of these papers are included herein in their entirety, by way of reference.

The Sox2 protein may be introduced into the cells being treated in combination with one or more other components of what is referred to herein as the “treatment agent”, including for example nucleic acids or proteins such as DNA methyl transferases, histone deacetylases, histones, nuclear lamins, transcription factors, activators, repressors, growth factors, hormones or cytokines as well as other agents such as detergents, salt solutions, compatible solvents, buffers, demethylating agents, nutrients or active compounds. However, it is preferred that the Sox2 protein or analogue or variant thereof is at least to some extent isolated or purified from other components of a cytoplasmic extract from which it may be obtained. Alternatively, in another alternative method, recombinant Sox2 may be secreted by cells after appropriate modification, for example by introducing a secretory signal into the sequence, or may be isolated from bacteria transfected with an Sox2 construct.

Preferably the Sox2 protein is introduced to cells to be treated in conjunction with HIV-TAT protein, preferably in the form of a fusion protein of Sox2-TAT, or in conjunction with other cell permeabilisation peptides such as Penetratin, or as analogues, variants or fragments thereof that retain ability to initiate pluripotency.

Throughout this specification the terms “isolated” and “purified” are intended to define that an agent is at least 50% by weight free from proteins, antibodies and naturally-occurring organic molecules with which it is endogenously associated. Preferably the agent is at least 75% and more preferably at least 90%, 95% or 99% by weight pure. A substantially pure agent may be obtained by chemical synthesis, separation of the agent from natural sources or production of the agent in a recombinant host cell that does not naturally produce the agent. Agents may be purified using standard techniques such as for example those described by Ausubel et al¹⁶, the disclosure of which is incorporated herein in its entirety by way of reference. The agent is preferably at least 2, 5 or 10 times as pure as the starting material from which it is derived, as measured using polyacrylamide gel electrophoresis, column chromatography, optical density, HPLC analysis or western analysis. Preferred methods of purification include immuno precipitation, column chromatography such as immuno affinity chromatography, magnetic bead immuno affinity chromatography and panning with a plate-bound antibody. In the case where the Sox2 protein is produced by recombinant technology, the protein may be purified by virtue of specific sequences incorporated into the protein, as, for example, through Nickel column affinity where the protein has 6 or more histidine amino acids incorporated into the sequence.

The treatment agent introduced into the cells to be treated may also include one or more other transcription agents or their functionally equivalent analogues, variants or fragments. Such transcription agents may include one or more of Oct4, Nanog, Lin28, Klf4 or c-myc, as for example referred to by Okita et al⁴. For example, the other transcription factors may be introduced into the cell in the same manner as Sox2 (as recombinant proteins, optionally altered to include additional sequences such as HIV-TAT), or may be introduced into the cells by transfection of the gene encoding these transcription factors.

As indicated above it is included within the invention to introduce not only Sox2 (or Sox2-TAT) or other transcription factors into the cells being treated to induce pluripotency, but also to introduce either in addition or in their place functionally equivalent analogues, variants or fragments. By the phrase “functionally equivalent” it is intended to convey that the variant, analogue or fragment is also effective in inducing pluripotency in the cells treated according to the invention and preferably a given quantity of the analogue, variant or fragment is at least 10%, preferably at least 30%, more preferably at least 50, 60, 80, 90, 95 or 99% as effective as an equivalent amount of Sox2 or the transcription factor from which the analogue, variant or fragment is derived. Determination of the relative efficacy of the analogue, variant or fragment can readily be carried out by utilising a prescribed amount of the analogue, variant or fragment in the methods of the invention and then comparing pluripotency achieved against the same amount of Sox2 protein or transcription factor from which the analogue, fragment or variant is derived. Quantification of pluripotency by cells treated in this regard can readily be determined by routine methods, as discussed above.

Analogues and variants are intended to encompass proteins having amino acid sequence differing from the protein from which they are derived by virtue of the addition, deletion or substitution of one or more amino acids to result in an amino acid sequence that is preferably at least 60%, more preferably at least 80%, particularly preferably at least 85, 90, 95, 98, 99 or 99.9% identical to the amino acid sequence of the original protein. The analogues or variants specifically include polymorphic variants and interspecies homologues. In particular, the term “variants” is intended to encompass the inclusion in the protein of additional functional sequences, such as the transcriptional activator sequence VP16 derived from the herpes simplex virus or the TAT sequence derived from the Human Immunodeficiency virus. It is also intended to encompass the deletion of sequences within the normal Sox2 sequences so as to alter the distribution and metabolism of the protein, such as, for example, PEST sequences associated with protein metabolism and destruction.

By reference to “fragments” it is intended to encompass fragments of a protein that are of at least 10, preferably at least 20, more preferably at least 30, 40 or 50 amino acids in length and which are functionally equivalent to the protein of which they are a fragment.

Throughout this specification the terms “polypeptide”, “peptide” and “protein” are used interchangeably to refer to a polymer of amino acid residues. The terms apply equally to amino acid polymers in which one or more amino acid residues is an artificial chemical mimetic of a corresponding naturally occurring amino acid, as well as to both naturally and non-naturally occurring amino acid polymers.

The term “amino acid” refers to naturally occurring and synthetic amino acids as well as amino acid analogues and amino acid mimetics that function in a manner similar to the naturally occurring amino acids. Naturally occurring amino acids are those encoded by the genetic code, as well as those amino acids that are later modified, e.g., hydroxyproaline, gamma-carboxyglutamate, and O-phosphoserene. “Amino acid analogues” refers to compounds that have the same basic chemical structure as a naturally occurring amino acid, that is a carbon that is bound to a hydrogen, a carboxyl group, an amino group and an R group, e.g., homoserene, norlusene, methianene sulfoxide and methanene methyl sulphonian. Such analogues have modified R groups (e.g. norlusene) or modified peptide backbones, but retain the same basic chemical structure as a naturally occurring amino acid. Amino acid mimetics refers to chemical compounds that have a structure that is different from the general chemical structure of an amino acid, but retain a function similar to that of a naturally occurring amino acid.

The methods of the invention can also involve introduction into the cells of growth factors or growth promoting agents suitable for the maintenance of pluripotency. For example, such growth factors may include members of the fibroblast growth factor family (such as in particular FGF4) as well as insulin-like growth factors and epidermal growth factors. Growth factors or growth promoting agents suitable for the maintenance of pluripotency may of course also be included within the treatment agent.

By the phrase “suitable to maintain pluripotency” it is intended to mean culturing the cells in media, growth factors and other media additives, with or without the use of suitable feeder layer cells (such as mouse embryonic fibroblasts) that have been shown to maintain the pluripotent state in cultured ES cells.

The Sox2-TAT protein or functionally equivalent analogue or variant thereof enters target cells unaided by virtue of the TAT cell permeable peptide, but Sox2 may alternatively be introduced into the cells to be treated according to the invention by a variety of different means. For example, the treatment agent to be introduced into the cells can be introduced by utilising detergent, bacterial toxin or electroporation techniques for increasing permeabilisation of the cell. To some extent these methods introduce repairable pores, voids or weaknesses into the cellular membrane which allow the agents to pass across the membrane. An example of a detergent that may be utilised to achieve permeabilisation is digitonin, and streptolysin 0 is a bacterial toxin commonly used in this manner. Electroporation of a plasma membrane is a technique commonly used for introduction of foreign DNA during cell transfections, but can also be used for introduction of proteins. This method introduces large size and temporary openings in the plasma membrane which allows free diffusion of extra-cellular components into the cells, without the requirement for active uptake. Electroporation parameters may be tested and optimised for the specific type of cell being treated and the particular protein or proteins being introduced. Electroporation techniques are well known in the art and are further described in detail in Sambruck & Russell¹¹. Another agent that may be utilised in assisting introduction of proteins or other agents into the cells is the BioPorter® protein delivery reagent (Gene Therapy Systems, Inc.) which is a unique lipid based formulation that allows the delivery of proteins, peptides or other bioactive molecules into a broad range of cell types. It interacts non-covalently with the protein creating a protective vehicle for immediate delivery into cells. The reagent fuses directly with the plasma membrane of the target cell. The extent of introduction can be monitored by TRITC-conjugated antibody uptake during the treatment. This is easily detected using low light fluorescence on living cells. Molecules that have been successfully introduced in this manner into various cell types include high and low molecular weight dextran sulphate, β-galactasidase, caspase 3, caspase 8, granzime B and fluorescent antibody complexes.

Examples of cell-permeant peptide vectors that may be utilised to introduce agents into cells include antennapedia/penetratin, TAT and signal-peptide based sequences as further discussed in Ford et al¹⁷, the disclosure of which is included herein in its entirety by way of reference.

A further specific technique that may be utilised in introducing agents into the cells to be treated is Pro-Ject™ transfection using Pro-Ject™ reagent (Pierce, Rockford Il., USA). Pro-Ject™ is a cationic lipid-based carrier system that can be used to deliver biologically active proteins, peptides or antibodies into cells. Pro-Ject™ Reagent/protein complexes attach to negatively charged cell surfaces and enter the cell either by directly fusing with the plasma membrane or by endocytosis and subsequent fusion with the endosome.

The amount of Sox2 protein or its analogues, variants or fragments introduced into the cells in which pluripotency is intended to be initiated and which is effective for the induction of pluripotency, can readily be optimised by persons skilled in the art. The effective amount will, however, vary depending upon the technique adopted for introducing the agent into the cells and may also depend upon the types and species of cell utilised, cell culture conditions, use of other transcription factors and indeed whether the method is conducted in vivo or in vitro. In determining the effective amount of the cells or treatment agent to be administered in the methods of treatment or prophylaxis according to the invention the physician can readily conduct an appropriate dose response trial which evaluates the efficacy of the treatment as well as taking into consideration issues such as toxicities, transplantation reactions, progression of the disease, and the like. However, as a general guide effective amounts for inducing pluripotency within the cell of Sox2 protein or functionally equivalent analogue, variant or fraction thereof may fall within the range of 0.01-10 μg/ml per 10⁵ target cells. Administrations according to the invention can be accomplished via single or divided doses.

As mentioned above, patients may for example be treated in an in vivo or indeed an in vitro fashion. By in vivo treatment it is intended to mean that methods of initiating pluripotency in mammalian cells are conducted upon these cells while they are located within the organism concerned. In relation to in vitro applications of the treatment methods it is intended to convey that mammalian cells, preferably those derived from an organism of the same species, and particularly preferably derived from the particular patient concerned, are exposed to the treatments according to the invention in an in vitro or cell culture setting. After exposure of the cells to the treatment agent to induce pluripotency the cells so treated, or progeny cells ultimately derived from them, are treated to induce differentiation along desired lineages before being returned to the patient. Cells can readily be removed from patients for conducting in vitro aspects of the invention by routine techniques such as by biopsy of the appropriate tissue or organ or extraction of cell containing fluid from the patient. The cells obtained can then be cultured under appropriate cell culture conditions, as will be further explained. Similarly, cells in which pluripotency has been initiated and which have then been differentiated along desired paths can be introduced to the patient by a variety of conventional means, such as for example by intravenous, intra-arterial, intramuscular, transdermal, intraperitoneal or direct injection into an organ using a physiologically compatible suspension of the treated cells. It is also possible to surgically implant the cells into a desired location within the organism, possibly by utilising endoscopic techniques to minimise patient trauma.

In in vivo embodiments of the invention the treatment agent may similarly be exposed to the cells into which it is intended to be introduced by a variety of conventional means. For example, the treatment agent, possibly including one or more physiologically compatible permeabilisation agents, may be injected into the appropriate tissue or organ, may be applied to the heart or another tissue or organ using a patch or matrix or may be applied or injected to a suitable tissue or organ in conjunction with a liposomal delivery system. Indeed, specific endogenous cells within the patient may be subjected to electroporation permeabilisation to assist in cellular uptake of the treatment agent. For example, techniques and agents previously mentioned in the context of introducing the treatment agent into the cells to be treated may similarly be utilised for in vivo treatments, where these methods or agents are physiologically compatible and do not present an undue risk to general patient health. Naturally, the general state of health, sex, weight, age and pregnancy status of the patient would be considered by the skilled medical practitioner administering the treatment when optimising the particular treatment to meet individual patient needs.

In conjunction with in vivo aspects of the invention it is possible to conduct surgical or other intervention before or after exposure of cells to the treatment agent. For example, it is possible to relocate myoblast cells to a damaged region of the heart either before or after exposure to the treatment agent.

Further details on the formulation of injectable formulations which can be utilised for preparation of injectable cell suspensions and treatment agents, as well as preparation of other pharmaceutical forms for delivery of treatment agents according to the invention are explained in detail in Remington's Pharmaceutical Sciences¹⁸, the disclosure of which is included herein in its entirety by way of reference. As will be understood, pharmaceutically acceptable carriers and formulations are determined in part by the particular agent, compound or composition being administered (e.g., the cell or treatment agent), as well as by the particular method used to administer the formulation. In the case of in vivo administration of cells together with the treatment agent, the carriers can include slow release agents that deliver a dose of the treatment agent to the cells in a controlled fashion over time (hours, days or weeks as necessary). Such controlled release carriers include polymers, lipid formulations, and other biodegradable or non-biodegradable materials.

Formulations suitable for parenteral administration, such as, for example, by intravenous, intramuscular, intradermal, intraperitoneal, and subcutaneous routes, include aqueous and non-aqueous, isotonic sterile injection solutions, which can contain physiologically acceptable (especially pharmaceutically acceptable) carriers and diluents such as antioxidants, buffers, bacteriostats, and solutes that render the formulation isotonic with the blood of the intended recipient, and aqueous and non-aqueous sterile suspensions that can include suspending agents, solubilisers, thickening agents, stabilisers, and preservatives. In the practice of this invention, compositions can be administered, for example, by direct surgical transplantation, intraportal administration, intravenous infusion, or intraperitoneal infusion.

Injection solutions and suspensions can be prepared from sterile powders, granules, and tablets. The dose of cells or treatment agent administered to a patient, in the context of the present invention should be sufficient to effect a beneficial therapeutic response in the patient over time. The dose will be determined by the efficacy of the particular cells or treatment agent employed and the condition of the patient, as well as the body weight or surface area of the patient to be treated. The size of the dose also will be determined by the existence, nature, and extent of any adverse side-effects in a particular patient.

Through inducing pluripotency within particular cell populations that are either within or are introduced into a mammalian (preferably human) patient the methods of the invention can be adopted for the treatment and/or prevention of degenerative disease or injury. Examples of such degererative diseases and injuries include Alzheimer's disease, Parkinson's disease, multiple sclerosis, motor neurone disease, diabetes mellitus, stroke, cardiovascular disease, spinal cord or other neuronal injury, surgical damage, radiation damage, muscular injury, muscular dystrophy, skin injury, bone injury, burns, osteoporosis, vascular disease or injury and trauma.

This invention relies upon routine techniques in the field of cell culture, and suitable methods can be determined by those of skill in the art using known methodology (see, e.g., Freshney et al¹⁹). In general, the cell culture environment includes consideration of such factors as the substrate for cell growth, cell density and cell contact, the gas phase, the medium and temperature.

In a preferred embodiment, the cells are grown in suspension as three dimensional aggregates. Suspension cultures can be achieved by using, e.g., a flask with a magnetic stirrer or a large surface area paddle, or on a plate that has been coated to prevent the cells from adhering to the bottom of the dish. In a preferred embodiment, the cells are grown in Costar dishes that have been coated with a hydrogel to prevent them from adhering to the bottom of the dish.

For the cells of the invention that are cultured under adherent conditions, plastic dishes, flasks, roller bottles, or microcarriers in suspension are used. Other artificial substrates can be used such as glass and metals. The substrate is often treated by etching, or by coating with substances such as collagen, chondronectin, fibronectin, and laminin. The type of culture vessel depends on the culture conditions, e.g., multi-well plates, petri dishes, tissue culture tubes, flasks, roller bottles, and the like.

Cells are grown at optimal densities that are determined empirically based on the cell type. For example, a typical cell density for .beta.lox5 cultures varies from 1×10³ to 1×10⁷ cells per ml. Cells are passaged when the cell density is above optimal.

Cultured cells are normally grown in an incubator that provides a suitable temperature, e.g., the body temperature of the animal from which is the cells were obtained, accounting for regional variations in temperature. Generally, 37° C. is the preferred temperature for cell culture. Most incubators are humidified to approximately atmospheric conditions.

Important constituents of the gas phase are oxygen and carbon dioxide. Typically, atmospheric oxygen tensions (20%) are used for cell cultures, though for some cell types lower oxygen concentrations of 10%, 5% or 2% are preferred. Culture vessels are usually vented into the incubator atmosphere to allow gas exchange by using gas permeable caps or by preventing sealing of the culture vessels. Carbon dioxide plays a role in pH stabilisation, along with buffer in the cell media and is typically present at a concentration of 1-10% in the incubator. The preferred CO₂ concentration typically is 5%.

Defined cell media are available as packaged, premixed powders or presterilised solutions. Examples of commonly used media include DME, RPMI 1640, Iscove's complete media, or McCoy's Medium (see, e.g., GibcoBRL/Life Technologies Catalogue and Reference Guide; Sigma Catalogue). Typically, low glucose DME or RPMI 1640 are used in the methods of the invention. Defined cell culture media are often supplemented with 5-20% serum, typically heat inactivated, e.g., human, horse, calf, and fetal bovine serum. Typically, 10% fetal calf serum or human serum is used in the methods of the invention. The culture medium is usually buffered to maintain the cells at a pH preferably from 7.2-7.4. Other possible supplements to the media include, e.g., antibiotics, amino acids, sugars, and growth factors such as hepatocyte growth factor/scatter factor (HGF), Insulin-like growth factor-1 (IGF-1), members of the fibroblast growth factor (FGF) family, members of the bone morphogenic protein (BMP) family, and epidermal growth factor (EGF).

It is also to be understood that the Sox2 or other transcription factors or their functionally equivalent analogues or variants that may comprise or be included within the treatment agent may be chemically synthesised, recombinantly produced or isolated from mammalian cells. Chemical synthesis, recombinant production and isolation techniques that may be adopted are well recognised in the art, as for example outlined in Ausubel et al¹⁴ and Sambruck & Russell¹¹.

It is to be recognised that the present invention has been described by way of example only and that modifications and/or alterations thereto which would be apparent to persons skilled in the art, based upon the disclosure herein, are also considered to fall within the spirit and scope of the invention.

The invention will now be further described with reference to the following non-limiting examples.

### EXAMPLES

### Example 1

### Production and Purification of Sox2 and Sox2-TAT Proteins

### Materials and Methods

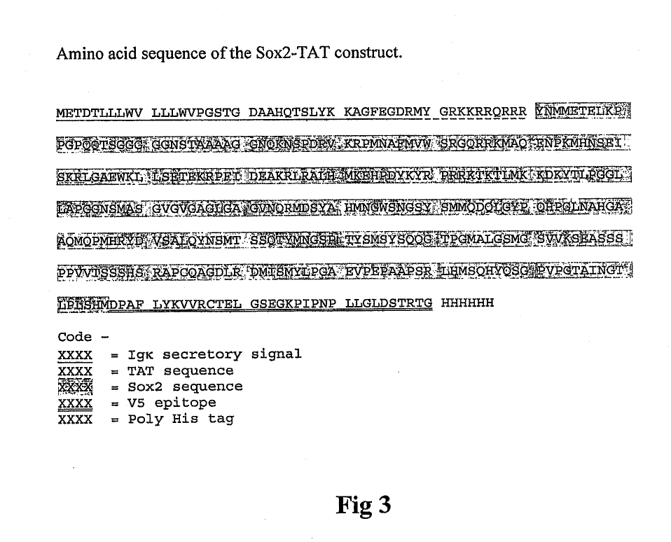

The sequence of human Sox2 was cloned and sequenced from an embryonic stem cell cDNA library using the PCR primers shown in Table 1. A second variant was then made by fusing the HIV-TAT sequence to the 3′ end of the Sox2 sequence, using the PCR primers shown in Table 1. The Sox2-TAT clone was then inserted into the pSecTag/FRT/V5-His-TOPO vector using standard methods (shown in SEQ ID No. 7 and FIG. 3). This vector includes an Igκ secretory signal, allowing the protein to be secreted into the medium. The recombinant proteins also have a V5 tag to allow identification and tracking of the protein, and a His sequence to enable purification on a nickel column. The Sox2-TAT clone was then stably transfected into chinese hamster ovary (CHO) cells.

TABLE 1
Sox2 Primer Sequences

| | Primer |
|---|---|
| Sox2 Forward | gcaggcttcgaaggagatagaaccatgta |
| | caacatgatggagacggag |
| Sox2-TAT Forward | gatagaatgtatggcaggaagaagcggag |
| | acagcgacgaagatacaacatgatggaga |
| | cggag |
| Sox2 and Sox2-TAT | ggggaccactttgtacaagaaagctgggt |
| Reverse | ccatgtgtgagaggggcagtgt |

### Recombinant Protein Isolation

Stably transfected Sox2-TAT CHO clones were expanded in RPMI medium containing 2% FCS, 0.5 mg/ml Fetuin, and 0.5 mg/ml bovine serum albumin (BSA). When the cells were 80% confluent they were harvested using trypsin/EDTA, washed then lysed using NePer lysis buffer (Invitrogen). The cell lysate was then loaded onto a nickel column (Talon), which was then washed with 10 ml of wash buffer. Recombinant protein was then eluted from the column using elution buffer (300 ug/ml imidazole). The washes and purified protein were then run on an SDS PAGE gel and transferred to a nylon membrane. Protein was detected using anti-V5 antibodies (1:1000) labeled with biotin.

### Results

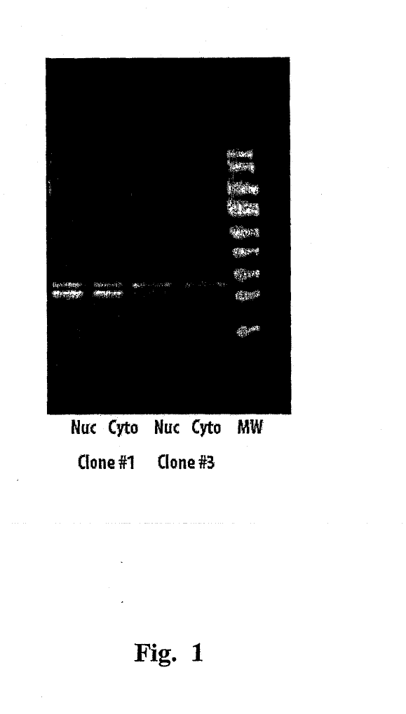

FIG. 1 shows the Western Blot, which demonstrates that purified recombinant Sox2-TAT protein was isolated in the eluate

### Example 2

### Functionality of the Recombinant Sox2 Protein

### Materials and Methods

A luciferase reporter was used to quantitate functionality of the recombinant protein. The promoter sequence for Nanog, a downstream target of Sox2, was amplified from genomic DNA by PCR using the primers shown in Table 2. The full Nanog promoter sequence is shown in SEQ 8. This was then inserted into pGL reporter vector (Invitrogen), which includes a luciferase sequence downstream of the inserted promoter. The vector was then amplified in bacteria E. coli, isolated and then transfected into CHO cell lines stably transfected with the Sox2-TAT construct. Luminescence was measured 48 hours later using a Tecan luminometer.

TABLE 2
Nanog promoter primers

| | Primer |
|---|---|
| Nanog promoter | cgcggtaccgatgggcacggagtagtcttg, |
| Forward | |
| Nanog promoter | gttagtatagaggaagaggagctcgaggcg |
| Reverse | |

### Results

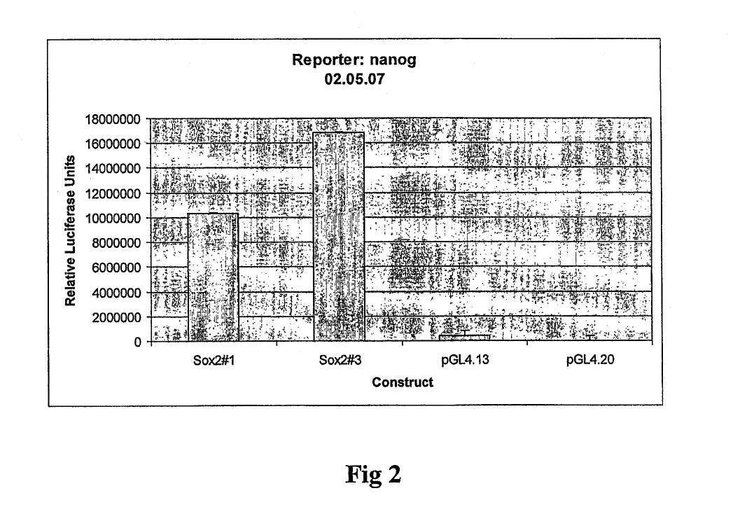

FIG. 2 shows the expression of luciferase in two subclones of CHO cells stably transfected with the Sox2-TAT construct. The levels of luciferase expression are considerably greater than those seen in the positive control (pGL4.13), indicating a high level of activity of the recombinant protein.

### ABBREVIATIONS

BMP Bone morphogenic protein
DNA Deoxyribose nucleic acid
DME Dulbecco's modified Eagles' medium
EGF Epidermal growth factor

### ES Embryonic Stem

FGF Fibroblast growth factor
FCS Fetal calf serum
FRT Flipase recognition target
HGF Hepatocyte growth factor

### His Histidine

HIV Human immunodeficiency virus
HMG High mobility group
HOX Homeobox cluster gene
IGF-1 Insulin-like growth factor 1

### IgG Immunoglobulin G

Igκ Immunoglobulin kappa light chain
iPS Induced pluripotent stem
Klf4 Krüppel-like factor 4
Oct4 Octamer-binding protein 4, also known as POU domain, class 5, transcription factor 1
POU Pit-Oct-Unc transcription factor family

### RPMI Roswell Park Memorial Institute

RT-PCR Real time polymerase chain reaction

### Sox2 SRY-related HMG box 2

SRY Sex-determining region Y

### TAT Transactivator

### TOPO Topoisomerase

TRITC Tetramethyl rhodamine isothiocyanate

### REFERENCES

1. http://stemcells.nih.gov/info/scireport/2001report.htm
2. Kume S. Stem-cell-based approaches for regenerative medicine. Develop. Growth Differ. (2005) 47, 393-402
3. Takahashi K, Yamanaka S. Induction of pluripotent stem cells from mouse embryonic and adult fibroblast cultures by defined factors. Cell. 2006 Aug. 25; 126(4):663-76.
4. Okita K, Ichisaka T, Yamanaka S. Generation of germline-competent induced pluripotent stem cells. Nature. Published online 6 Jun. 2007
5. Wernig M, Meissner A, Foreman R, Brambrink T, Ku M, Hochedlinger K, Bernstein B. E., Jaenisch R. In vitro reprogramming of fibroblasts into a pluripotent ES-cell-like state Nature. Published online 6 Jun. 2007
6. Takahashi K, et al, induction of pluripotent stem cells from adult human fibroblasts by defined factors. Cell (2007), doi:10.1016/j.cell.2007.11.019
7. Yu J et al. Induced pluripotent stem cell lines derived from human somatic cells. Sciencexpress (2007) science.1151526
8. Pan G J, Chang Z Y, Scholer H R, Pei D Stem cell pluripotency and transcription factor Oct4. Cell Res. 2002 December; 12(5-6):321-9.
9. Kamachi, Y., Uchikawa, M., and Kondoh, H. 2000. Pairing SOX off. Trends Genet. 16: 182-187
10. Horb M E, Shen C-N, Tosh D, Slack JMW Experimental Conversion of Liver to Pancreas. Curr Biol 2003:13; 105-115
11. Yamada Y, Sakurada K, Takeda Y, Gojo S, Umezawa A. Single-cell-derived mesenchymal stem cells overexpressing Csx/Nkx2.5 and GATA4 undergo the stochastic cardiomyogenic fate and behave like transient amplifying cells. Exp. Cell Res. 2007:313(4):698-706
12. Prochiantz A, Joliot A. Can transcription factors act as cell-cell signaling molecules? Nature Mol Cell Biol 2003; 4:814-819
13. Sambruck & Russell, Molecular Cloning: A laboratory manual, 3rd Edition, 2001, Cold Spring Harbour Laboratory Press, New York.
14. Stevanovic M., Zuffardi O., Collignon J., Lovell-Badge R., Goodfellow P.; The cDNA sequence and chromosomal location of the human SOX2 gene. Mamm. Genome 5:640-642 (1994).
15. Boiani M, Scholer H R Regulatory networks in embryo-derived pluripotent stem cells. Nat Rev Mol Cell Biol. 2005 November; 6(11):872-84.
16. Ausubel et al, Current Protocols in Molecular Biology, John Wiley & Sons, New York, 2000.
17. Ford K G, Souberbielle B E, Darling D, Farzaneh F. Protein transduction: an alternative to genetic intervention? Gene Therapy (2001) 8, 1-4
18. Remington's Pharmaceutical Sciences, 18th Ed., Mack Publishing Co., Easton, Pa., USA
19. Freshney et al., Culture of Animal Cells (e. sup. rd. ed. 1994).

1. A method of inducing pluripotency in a responsive mammalian cell, which comprises introducing into the cell an effective amount for initiating pluripotency within the cell of Sox2 protein or a functionally equivalent analogue, variant or fragment thereof.

2. (canceled)

3. (canceled)

4. (canceled)

5. A method of treatment and/or prophylaxis of a degenerative disease or injury in a mammal, which comprises introducing into responsive cells of the mammal an effective amount of Sox2 protein or a functionally equivalent analogue, variant or fragment thereof.

6. (canceled)

7. The method of claim 5 wherein the degenerative disease or injury is selected from the group consisting of Alzheimer's disease, Parkinson's disease, multiple sclerosis, motor neurone disease, diabetes mellitus, stroke, cardiovascular disease, spinal cord or other neuronal injury, surgical damage, radiation damage, muscular injury, Muscular dystrophy skin injury, bone injury, burns, osteoporosis, vascular disease or injury and trauma.

8. The method of claim 1 wherein the Sox2 protein or a functionally equivalent analogue, variant or fragment thereof is introduced into the cells in conjunction with one or more other transcription factors.

9. The method of claim 1 wherein the Sox2 protein or a functionally equivalent analogue, variant or fragment thereof is introduced into the cells in conjunction with one or more other transcription factors selected from Oct4, Nanog, Lin28, Klf4 or c-myc, or their functionally equivalent analogues, variants or fragments.

10. The method of claim 8, wherein the one or more other transcription factors or their functionally equivalent analogues, variants or fragments are introduced into the cells in the form of recombinant protein.

11. The method of claim 8, wherein the one or more other transcription factors or their functionally equivalent analogues, variants or fragments are introduced into the cells by transfection into the cells of functional genes encoding for the transcription factors or their functionally equivalent analogues, variants or fragments.

12. The method of claim 1 wherein one or more growth factors or growth promoting agents suitable for maintaining pluripotency are also introduced into the cell/s.

13. The method of claim 1 wherein one or more growth factors or growth promoting agents suitable for maintaining pluripotency are also introduced into the cell/s and wherein the one or more growth factors are fibroblast growth factors, insulin-like growth factors and/or epidermal growth factors.

14. The method of claim 12 wherein the one or more growth factors comprises FGF4.

15. (canceled)

16. (canceled)

17. The method of claim 1 wherein the responsive mammalian cells are mammalian cells other than pluripotent stem cells.

18. The method of claim 1 wherein the responsive mammalian cell is selected from one or more of hepatocytes, fibroblasts, endothelial cells, B cells, T cells, dendritic cells, keratinocytes, adipose cells, epithelial cells, epidermal cells, chondrocytes, cumulus cells, neural cells, glial cells, astrocytes, cardiac cells, oesophageal cells, skeletal muscle cells, skeletal muscle satellite melanocytes, hematopoietic cells, osteocytes, macrophages, monocytes, mononuclear cells or stem cells including embryonic stem cells, embryonic germ cells, adult brain stem cells, epidermal stem cells, skin stem cells, pancreatic stem cells, kidney stem cells, liver stem cells, breast stem cells, lung stem cells, muscle stem cells, heart stem cells, eye stem cells, bone stem cells, spleen stem cells, immune system stem cells, cord blood stem cells, bone marrow stem cells and peripheral blood stem cells.

19. (canceled)

20. (canceled)

21. (canceled)

22. (canceled)

23. (canceled)

24. (canceled)

25. An agent for inducing pluripotency in a responsive mammalian cell, which comprises Sox2 protein or a functionally equivalent analogue, variant or fragment thereof, one or more other transcription factors and one or more physiologically acceptable carriers and/or diluents.

26. The agent of claim 25 further comprising one or more other transcription factors.

27. The agent of claim 26 wherein the one or more other transcription factors are selected from Oct4, Nanog, Lin28, Klf4 and c-myc, or their functionally equivalent analogues, variants or fragments.

28. (canceled)

29. The agent of claim 25 further comprising one or more growth factors or growth promoting agents suitable for maintaining pluripotency.

30. (canceled)

31. The agent of claim 25 comprising Sox2-TAT protein.

32. The method of claim 5, which comprises removing the responsive cells from the mammal, culturing the responsive cells in a suitable medium, introducing into the responsive cells an effective amount of Sox2 protein or a functionally equivalent analogue, variant or fragment thereof and subsequently returning the responsive cells to the mammal.

33. The method of claim 1, wherein a Sox2-TAT protein or a functionally equivalent analogue, variant or fragment thereof is introduced into the cells in conjunction with one or more other transcription factors selected from Oct4, Nanog, Lin28, Klf4 or c-myc, or their functionally equivalent analogues, variants or fragments and wherein the mammalian cell is a human cell.


SEQUENCE LISTING (13 sequences)

SEQ ID NO: 1 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 2 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 3 - protein, 305 aa, Homo sapiens
MSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 4 - protein, 470 aa, Homo sapiens
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 5 - protein, 439 aa, Homo sapiens
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 6 - protein, 209 aa, Homo sapiens
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 7 - protein, 406 aa, Artificial Sequence
  Recombinant protein
METDTLLLWVLLLWVPGSTGDAAHQTSLYKKAGFEGDRMYGRKKRRQRRRYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHMDPAFLYKVVRCTELGSEGKPIPNPLLGLDSTRTGHHHHHH

SEQ ID NO: 8 - DNA, 1176 bp, Homo sapiens
gatgggcacggagtagtcttgaaagacatgacaaatcaccagacctgggaagaagctaaagagccagagggaaaaagccagaagtcgactacctgggaggagggatagacaagaaaccaaactaaaggaaactaaggtaggtgctgaaaacaagtaccattttcaacattaactgatgccttggcttcatgctataatgccatgttgtgtttcactataacctcagagtgaatgaaagaggaaaatggagctagttgaaatttctgcctaaactagccagattttgagacactaagttatctcaaatcaagaaatcaccctaatgagaatttcaataacctcaggaatttaaggtgcatgcatcccccaccccccccttttttttttgagacgtagtcccgctctgttgcccaggctggagtacagtggcgcgatatcggctcaccacaacctctgcctcccaggttcaagggattctcccgcctcagcttccagagtagctgggactacagacacccaccaccatgcgtggctaatttttgtatttttagtagagagggggtttcgccatgttggccaggctggtttcaaactcctgacttcaggtgatccgcctgccacggcctcccaatttactgggattacaggggtgggccaccgcgcccggcctttttcttaatttttaaaaatattaaagttttatcccattcctgttgaaccatattcctgatttaaaagttggaaacgtggtgaacctagaagtatttgttgctgggtttgtcttcaggttctgttgctcggttttctagttccccacctagtctgggttactctgcagctacttttgcattacaatggccttggtgagactggtagacgggattaactgagaattcacaagggtgggtcagtagggggtgtgcccgccaggaggggtgggtctaaggtgatagagccttcattataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaac

SEQ ID NO: 9 - DNA, 48 bp, Artificial sequence
  Sox2 forward primer
gcaggcttcgaaggagatagaaccatgtacaacatgatggagacggag

SEQ ID NO: 10 - DNA, 63 bp, Artificial sequence
  Sox2-tat forward primer
gatagaatgtatggcaggaagaagcggagacagcgacgaagatacaacatgatggagacggag

SEQ ID NO: 11 - DNA, 51 bp, Artificial sequence
  Synthetic
ggggaccactttgtacaagaaagctgggtccatgtgtgagaggggcagtgt

SEQ ID NO: 12 - DNA, 30 bp, Artificial sequence
  Nanog forward primer
cgcggtaccgatgggcacggagtagtcttg

SEQ ID NO: 13 - DNA, 30 bp, Artificial sequence
  Nanog reverse primer
gttagtatagaggaagaggagctcgaggcg